(19) United States
(12) Patent Application Publication
(10) Pub. No.: US 2024/0389948 A1
(43) Pub. Date: Nov. 28, 2024

(54) PREDICTION DEVICE, PREDICTION METHOD, AND PREDICTION PROGRAM

(71) Applicant: SHIMADZU CORPORATION, Kyoto-shi (JP)

(72) Inventors: Mikihiro YAMANAKA, Kyoto-shi (JP); Toshiaki NAKAMURA, Kyoto-shi (JP); Yusuke MURASE, Kyoto-shi (JP); Yutaro NAKATSU, Kyoto-shi (JP); Masaki KANAI, Kyoto-shi (JP); Masatoshi TAKAHASHI, Kyoto-shi (JP)

(21) Appl. No.: 18/290,685

(22) PCT Filed: Mar. 30, 2022
(86) PCT No.: PCT/JP2022/015821

§ 371 Date: Jan. 19, 2024

(30) Foreign Application Priority Data
Aug. 11, 2021 (JP) 2021-131058

(51) Int. Cl. A61B 5/00; A61B 5/22

(52) U.S. Cl. CPC A61B 5/7275; A61B 5/22

(57) ABSTRACT

A prediction device is equipped with a computing device and a communication device connected to the computing device in a communicable manner. The communication device acquires an AGEs measurement value of the subject a plurality of times in a time-series manner and acquires an SMI measurement value of the subject. The computing device calculates prediction information regarding an SMI prediction value, based on AGEs measurement values acquired by a plurality of times in a time-series manner and the SMI measurement value.

### TECHNICAL FIELD

The present disclosure relates to a prediction device, a prediction method, and a prediction program.

### BACKGROUND ART

Conventionally, a device for suggesting improvements to lifestyle habits based on an individual's health information is well known. For example, Patent Document 1 discloses a device that measures a subject's blood pressure, number of steps, or weight and suggests improvements to the subject's diet and exercise based on the measurement results.

### PRIOR ART DOCUMENT

### Patent Document

Patent Document 1: Japanese Unexamined Patent Application Publication No. 2020-160569

### SUMMARY OF THE INVENTION

### Problems to be Solved by the Invention

As for the information on an individual's health, in addition to blood pressure, step count, and weight, skeletal muscle mass (SMI: Skeletal Muscle mass Index, hereafter also referred to as “SMI”) can also be exemplified. In the diagnosis of sarcopenia, it is essential to measure a decrease in skeletal muscle mass as well as a decrease in walking speed and grip strength. Skeletal muscle mass declines with age or disease. SMI is expressed by a value obtained by dividing the total limb skeletal muscle mass by the square of the height, with a cutoff value for sarcopenia defined for each gender. Sarcopenia has received increasing attention in recent years as a factor contributing to physical dysfunction and risk of falls in the elderly. SMI of the quadriceps, which is a muscle in the human body that develops through exercise, can be improved by improving lifestyle habits, such as diet and exercise.

Thus, measuring SMI is important to observe whether lifestyle habits are improving. SMI is difficult to reflect in measurement values even if lifestyle habits are improved for several weeks, and it finally begins to reflect in measurement values after lifestyle habits are improved for several months. Therefore, when subjects attempted to improve their lifestyle habits while observing the SMI, they could not confirm the improvement effect unless they continued to improve their lifestyle habits for several months, making it difficult for them to maintain motivation to continue to improve their lifestyle habits.

The present disclosure has been made to solve such problems. The purpose of the present disclosure is to provide a technique to keep subjects motivated to continuously improve their lifestyle habits.

### Means for Solving the Problems

A prediction device according to one aspect of the present disclosure is provided with a computing device and a communication device connected to the computing device in a communicable manner. The communication device is configured to acquire a first measurement value of advanced glycation end-products of the subject a plurality of times in a time-series manner and acquire a second measurement value of the skeletal muscle mass of the subject. The computing device calculates prediction information regarding a prediction value of the second measurement value, based on the first measurement values acquired a plurality of times in a time-series manner and the second measurement value.

A prediction method according to another aspect of the present disclosure includes:

a step of acquiring a first measurement value of the advanced glycation end-products of the subject a plurality of times in a time-series manner;
a step of acquiring a second measurement value of the skeletal muscle mass of the subject; and
a step of calculating prediction information regarding a prediction value of the second measurement value, based on the first measurement value acquired a plurality of times in a time-series manner and the second measurement value.

A prediction program according to another aspect of the present disclosure is configured to make a computing device execute:

a step of acquiring a first measurement value of advanced glycation end-products of the subject a plurality of times in a time-series manner; and
a step of acquiring a second measurement value of the skeletal muscle mass of the subject; and
a step of calculating prediction information regarding a prediction value of the second measurement value, based on the first measurement value acquired a plurality of times in a time-series manner and the second measurement value.

### Effects of the Invention

According to the present disclosure, a subject can know prediction information regarding the prediction value of the SMI, based on the time-series changes in the measurement value in the advanced glycation end-products (hereinafter also referred to as “AGEs”), in addition to the SMI measurement value. With this, it is expected that it becomes easier to maintain motivation to continue improving lifestyle habits while observing the SMI.

### BRIEF DESCRIPTION OF THE DRAWINGS

FIG. 1 is a diagram showing a prediction system according to an embodiment.

FIG. 2 is a diagram showing a configuration of a prediction device according to an embodiment.

FIG. 3 is a diagram for explaining a user identification information table stored by the prediction device according to an embodiment.

FIG. 4 is a diagram for explaining a viewing information table stored by the prediction device according to an embodiment.

FIG. 5 is a diagram for explaining AGEs evaluation ranks.

FIG. 6 is a diagram showing one example of an AGEs measurement value.

FIG. 7 is a diagram for explaining SMI evaluation ranks.

FIG. 8 is a diagram for explaining the correlation between AGEs and SMI.

FIG. 9 is a diagram for explaining the correlation between AGEs and SMI.

FIG. 10 is a diagram showing one example of SMI prediction.

FIG. 11 is a diagram showing one example of a display screen at a user terminal according to an embodiment.

FIG. 12 is a diagram showing one example of a display screen at a user terminal according to an embodiment.

FIG. 13 is a diagram showing one example of a display screen at a user terminal according to an embodiment.

FIG. 14 is a diagram showing one example of a display screen at a user terminal according to an embodiment.

FIG. 15 is a diagram showing one example of a display screen at a user terminal according to an embodiment.

FIG. 16 is a flowchart of prediction processing executed by a prediction device of an embodiment.

### EMBODIMENTS FOR CARRYING OUT THE INVENTION

Hereinafter, some embodiments of the present disclosure will be described with reference to the attached drawings. Note that the same or equivalent part in the figures is assigned by the same reference symbol, and the description will not be repeated.

### <Configuration of Prediction System>

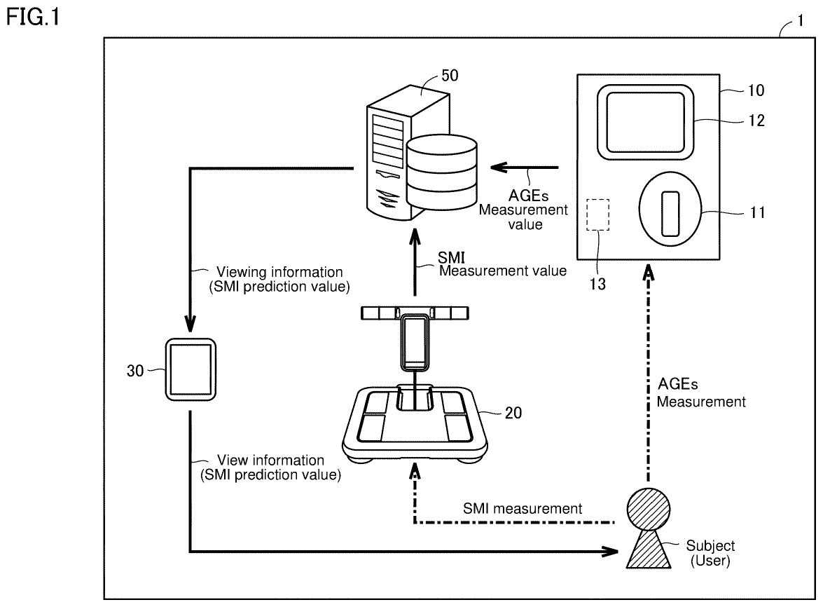

With reference to FIG. 1 to FIG. 4, a prediction system 1 according to an embodiment will be described. FIG. 1 is a diagram showing a prediction system 1 according to an embodiment. As shown in FIG. 1, the prediction system 1 is equipped with an AGEs measurement device 10, a SMI measurement device 20, a user terminal 30, and a prediction device 50.

The AGEs measurement device 10 is a device for measuring AGEs of a subject. The term “AGEs” collectively refers to several compounds, which are formed when sugars combine with proteins and react by oxidation, dehydration, and condensation. AGEs are believed to accumulate in the body as a result of disordered lifestyle habits, causing age-related and lifestyle-related diseases (e.g., diabetes, dementia). AGEs can change with shorter lifestyle improvements compared with SMI. AGEs can be reflected in AGEs measurement values by improving lifestyle habits for a few weeks. The subject includes an older adult who uses a nursing home, a person with age-related diseases, and a person with lifestyle-related diseases. The AGEs measurement device 10 is equipped with a measurement unit 11, a display 12, and a communication unit 13. Note that in this embodiment, the AGEs measurement device 10 is equipped with the display 12, but the display 12 may be omitted.

The measurement unit 11 non-invasively measures the AGEs measurement of the subject. Among a plurality of compounds contained in AGEs, there are compounds that have the property of emitting fluorescence upon being irradiated with a specific light. The measurement unit 11 measures the AGEs of the subject by utilizing the properties of these compounds.

When the subject touches the fingertip to the measurement unit 11, the measurement unit 11 emits light toward the skin from a light source (not illustrated). Note that the measurement unit 11 may also be configured to emit light toward the skin (e.g., arm) other than the fingertip of the subject. The light emitted by the measurement unit 11 is, for example, excitation light having a peak within a wavelength range of 410 nm or less. The measurement unit 11 receives the fluorescence excited by the light irradiated onto the skin with a light receiving element (not illustrated) and measures the degrees of AGEs accumulation based on the intensity of the received fluorescence. The display 12 displays the AGEs measurement results obtained by the measurement unit 11. The measurement results include, for example, the intensity of fluorescence received by the measurement unit 11 and the value obtained by converting the degree of AGEs accumulation into a score. Note that the measurement results may include a corrected value obtained by correcting the values derived from converting both the intensity of the fluorescence received by the measurement unit 11 and the degree of AGEs accumulation into a score.

The communication unit 13 transmits and receives data (information) to and from the prediction device 50 by wired or wireless communication. The communication unit 13 is an information terminal that can communicate with the prediction device 50 via a network, such as a desktop personal computer (PC), a laptop PC, a smartphone, a smartwatch, a wearable device, a tablet PC, and a network adapter. The communication unit 13 may be integrally configured with the display 12. The communication unit 13 may be separate from each of the measurement unit 11 and the display 12.

The SMI measurement device 20 is a device for measuring the SMI of a subject. The SMI measurement device 20 is, for example, a body composition meter, which may measure, in addition to the SMI, body weight, body mass index (BMI, hereinafter also referred to as “BMI”), body fat percentage, visceral fat level, basal metabolic rate, and body age.

The AGEs measurement device 10 and the SMI measurement device 20 are installed in various facilities such as pharmacies, medical institutions, nursing homes, and gyms. The AGEs measurement device 10 and the SMI measurement device 20 are assumed to be used solely by the subject, but may be managed by a supporter who supports the subject. When a supporter measures the AGEs of the subject they support using the AGEs measurement device 10, the AGEs measurement value (hereinafter also referred to as “AGEs measurement value”) is transmitted from the AGEs measurement device 10 to the prediction device 50. When a supporter measures the SMI of the subject they support using the SMI measurement device 20, the SMI measurement value (hereinafter also referred to as “the SMI measurement value”) is transmitted from the SMI measurement device 20 to the prediction device 50.

The supporter is a user of the service provided by the prediction system 1 (hereinafter also referred to as “information provision service”), like the subject, and is a person or an organization that supports the subject. For example, the supporter may be an employee of a daycare service facility (such as a day service and a daycare center), a staff member of a nursing home, a nurse, or a lifestyle counselor. As another example, a supporter may be a fitness gym instructor or a nutrition advisor. Further, the supporter may be a physician or a nurse in a hospital or a clinic. AGEs measurement values generally change over a period of one to several weeks, depending on the individual. Even in cases where the supporter does not communicate with the subject daily (e.g., in the case of about once a week), the supporter can obtain changes in AGEs by measuring the AGEs of the subject at that frequency. In contrast, SMI measurement values hardly change as quickly as AGEs measurement values within a few weeks, and they only begin to reflect improvements after several months of lifestyle changes.

The user terminal 30 is owned or used by the user. The user terminal 30 is an information terminal that can communicate with the prediction device 50 via a network, such as, a desktop PC, a laptop PC, a smartphone, a smartwatch, a wearable device, and a tablet PC. The user can obtain the information on the measurement results of AGEs and SMI of the subject stored in the prediction device 50 by accessing the prediction device 50 directly or indirectly using the user terminal 30.

The user is a user of an information provision service. Specifically, the user may be the subject or a supporter of the subject. Further, the user may be a family member, a relative, or a person related to the subject (e.g., an acquaintance) who has been authorized by the subject or the supporter to view the results of measurements on the subject.

The prediction device 50 is managed by a service provider offering an information provision service. Note that the service provider may be the manufacturer of the AGEs measurement device 10 who lends the AGEs measurement device 10 to the supporter. The prediction device 50 communicates with each of the AGEs measurement device 10, the SMI measurement device 20, and the user terminal 30 by functioning as a cloud computer.

In the prediction system 1 with the configuration described above, when a subject measures the AGEs using the AGEs measurement device 10, the AGEs measurement device 10 outputs the AGEs measurement values to the prediction device 50. Upon obtaining the AGEs measurement value from the AGEs measurement device 10, the prediction device 50 stores the obtained AGEs measurement value together with the subject's AGEs measurement values that were obtained in the past.

In the prediction system 1, when the subject measures the SMI using the SMI measurement device 20, the SMI measurement device 20 outputs the SMI measurement values to the prediction device 50. Upon obtaining the SMI measurement value from the SMI measurement device 20, the prediction device 50 stores the obtained SMI measurement values together with the subject's SMI measurement values that were obtained in the past.

Measuring AGEs and SMI is important to observe whether the subject's lifestyle habits are improving. Here, AGEs measurement values can reflect improvements in lifestyle habits within a few weeks, while SMI measurement values hardly reflect such improvements even after a few weeks of lifestyle habit improvements, and only start to show reflections after several months of sustained lifestyle improvements. Therefore, when subjects attempted to improve their lifestyle habits while observing the SMI, they could not confirm the improvement effect unless they continued to improve their lifestyle habits for several months, making it difficult for them to maintain motivation to continue improving their lifestyle habits.

Therefore, the prediction device 50 of the embodiment predicts the SMI measurement value based on the time-series changes in the AGEs measurement values that have been obtained and stored in the past and on the SMI measurement value that has been obtained and stored in the past, and stores the prediction value of the SMI measurement value (hereinafter also referred to as “SMI prediction value”). The prediction device 50 generates viewing information that can be viewed by the subject, the supporter, and the viewer based on the previously obtained AGEs measurement values, the previously obtained SMI measurement value, and the SMI prediction value.

Further, the prediction device 50 may generate viewing information based on other information about the subject. Other information includes, for example, blood pressure, body mass index (BMI), vegetable intake, walking speed, and grip strength. Further, the prediction device 50 may also analyze the health status of the subject based on the other information described above, generate analysis information including the analysis results, and include the analysis information in the viewing information. That is, the viewing information may include at least one of the following: the AGEs measurement values, the SMI measurement value, the SMI prediction value, as well as blood pressure, BMI, vegetable intake, walking speed, grip strength, and analysis information.

When a user requests viewing information using the user terminal 30, the prediction device 50 outputs the viewing information to the user terminal 30 in response to the request from the user terminal 30. The user terminal 30 displays the viewing information obtained from the prediction device 50.

This allows users to view the AGEs measurement values and the SMI measurement value obtained in the past using the user terminal 30, as well as to view the SMI prediction value calculated based on the time-series changes in the AGEs measurement values obtained in the past and the SMI measurement value obtained in the past.

### [Prediction Device Configuration]

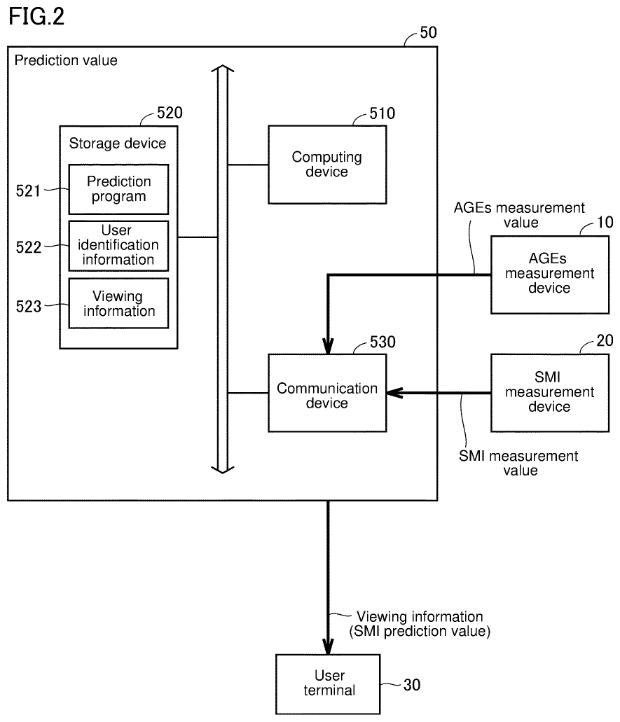

FIG. 2 is a diagram showing the configuration of the prediction device according to the embodiment. As shown in FIG. 2, the prediction device 50 is equipped with a computing device 510, a storage device 520, and a communication device 530.

The computing device 510 is one example of a computer and is a computing entity that executes various processing according to various programs. The computing device 510 includes at least one of, for example, a CPU (Central Processing Unit), an FPGA (Field Programmable Gate Array), a GPU (Graphics Processing Unit), and an MPU (Multi Processing Unit). Further, the computing device 510 may include volatile memory such as DRAM (Dynamic Random Access Memory) and SRAM (Static Random Access Memory), as well as nonvolatile memory such as ROM (Read Only Memory) and flash memory. Note that the computing device 510 may be configured by processing circuitry.

The storage device 520 includes a nonvolatile memory, such as an HDD (Hard Disk Drive) and an SSD (Solid State Drive). The storage device 520 stores various programs and data, including the prediction program 521 executed by the computing device 510, the user identification information 522 referenced by the computing device 510, and the viewing information 523.

The user identification information 522 includes information about the user, such as user ID, password, user name, age, and gender. The prediction device 50 can identify the user using the user identification information 522.

The viewing information 523 includes the AGEs measurement values, the SMI measurement value, and the SMI prediction value. Further, the viewing information 523 may include at least one of the following: blood pressure, BMI, vegetable intake, walking speed, grip strength, and analysis information.

Note that the storage device 520 may include a volatile memory, such as DRAM and SRAM, and a non-volatile memory, such as ROM and flash memory. Further, the computing device 510 may be equipped with a media reading device that is not illustrated. The computing device 510 may accept a removable disk, which is a storage medium, through a media reading device and may retrieve various programs and data, such as the prediction programs 521 and the corresponding user identification information 522, from the removable disk.

The communication device 530 is connected to the computing device 510 in a communicable manner. The communication device 530 transmits and receives data (information) with each of the AGEs measurement device 10, the SMI measurement device 20, and the user terminal 30 via wired or wireless communication.

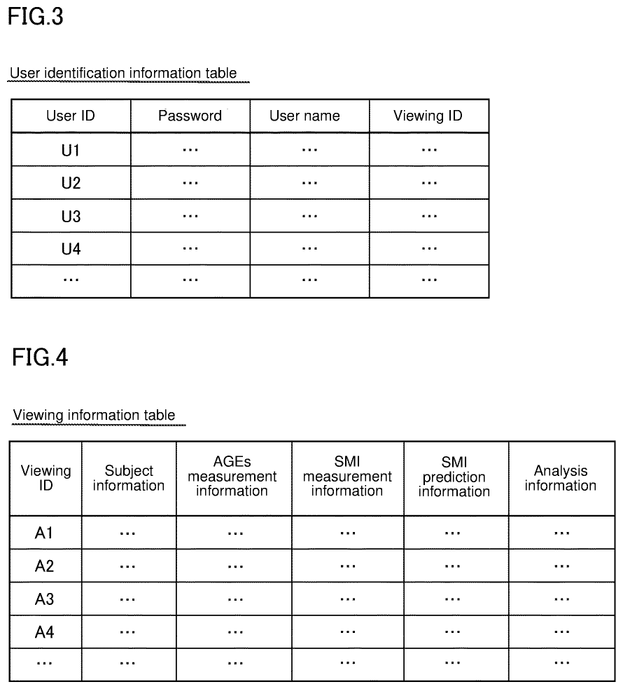

FIG. 3 is a diagram for explaining a user identification information table stored by the prediction device according to an embodiment. The prediction device 50 stores the user identification information 522 using the user identification information table in FIG. 3.

As shown in FIG. 3, the user identification information table stores information regarding the user ID, the password, the user name, and the viewing ID as the user identification information 522. Each user utilizing the information provision service is identified by the user identification information 522. For example, the first user is assigned “U1” as the user ID, and the second user is assigned “U2” as the user ID.

The user ID, the password, and the user name, among the user identification information 522, are entered by each user from the user terminal 30. The user terminal 30 outputs the input user identification information 522 to the prediction device 50. The prediction device 50 stores the user identification information 522 obtained from the user terminal 30 in the storage device 520 by storing it in the user identification information table.

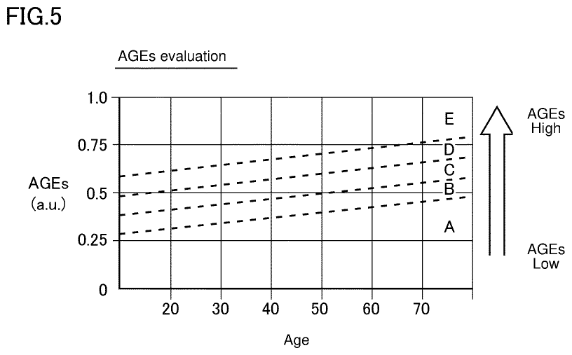

The viewing ID is associated with various types of viewing information, such as the subject information, the AGEs measurement information, the SMI measurement information, the SMI prediction information, and the analysis information in the viewing information table in FIG. 4, which will be described below. The prediction device 50 makes viewing information associated with a viewing ID available for viewing to the user, who is the owner of the user ID associated with the viewing ID.

FIG. 4 is a diagram for explaining the viewing information table stored by the prediction device 50 according to the embodiment. The prediction device 50 stores the viewing information 523 using the viewing information table in FIG. 4.

As shown in FIG. 4, the viewing information table includes viewing IDs, subject information, AGEs measurement information, SMI measurement information, SMI prediction information, and analysis information. The AGEs measurement information includes AGEs measurement values of the subject obtained in the past. The SMI measurement information includes an SMI measurement value of the subject obtained in the past. The SMI measurement information includes an SMI prediction value generated based on previously obtained AGEs measurement values and a previously obtained SMI measurement value, the SMI evaluation rank corresponding to the SMI measurement value as will described below, and a lifestyle advice based on the SMI prediction. The analysis information includes information regarding the results of the analysis of the health status of the subject.

### [Evaluation of AGEs]

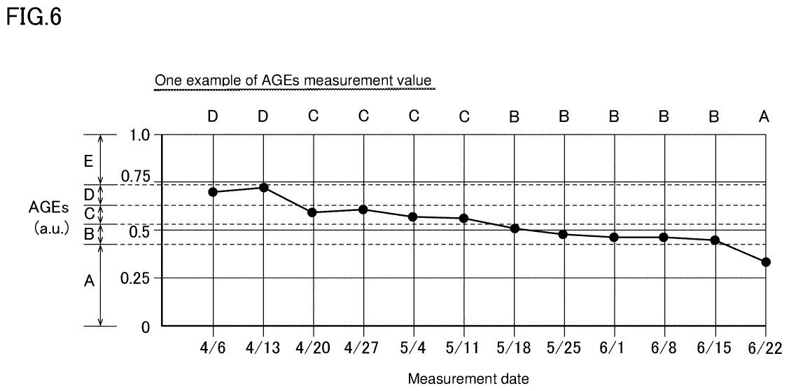

Referring to FIG. 5 and FIG. 6, the evaluation of AGEs will be described. FIG. 5 is a diagram for explaining AGEs evaluation ranks.

In FIG. 5, a graph is shown with the age of the subject on the horizontal axis and the AGEs measurement value on the vertical axis. As shown in FIG. 5, the prediction device 50 establishes a plurality of reference values for AGEs measurement values in a stepwise manner and ranks AGEs based on a comparison between AGEs measurement values and the plurality of reference values. For example, five ranks from A to E (hereinafter also referred to as “AGEs evaluation ranks”) have been established as the ranks assigned to AGEs measurement values. The AGEs evaluation rank of “A” gives the best AGEs measurement value evaluation, and the AGEs evaluation rank of “E” gives the worst AGEs measurement value evaluation.

The reference values for AGEs evaluation ranks shown in FIG. 5 are examples, and any value may be set as a reference value. Further, the reference value for the AGEs evaluation rank may not be limited to the age of the subject but may differ based on gender.

FIG. 6 is a diagram showing one example of AGEs measurement values. The prediction device 50 ranks the acquired AGEs measurement values based on the criteria shown in FIG. 5 and stores the AGEs evaluation ranks along with the AGEs measurement values as AGEs measurement information.

For example, in FIG. 6, a graph showing the time-series change in the AGEs measurement values of a subject is shown in which the horizontal axis represents the date and the vertical axis represents the AGEs measurement values. As shown in FIG. 6, the prediction device 50 assigns an AGEs evaluation rank from A to E to each AGEs measurement value when the subject's AGEs measurement value is obtained every week over a period of 4/6 to 6/22. In the case of this measurer, the AGEs measurement value gradually decreased (improved) from 4/6 to 6/22, and the AGEs evaluation rank is also improved from the initial D to A.

### [Evaluation of SMI]

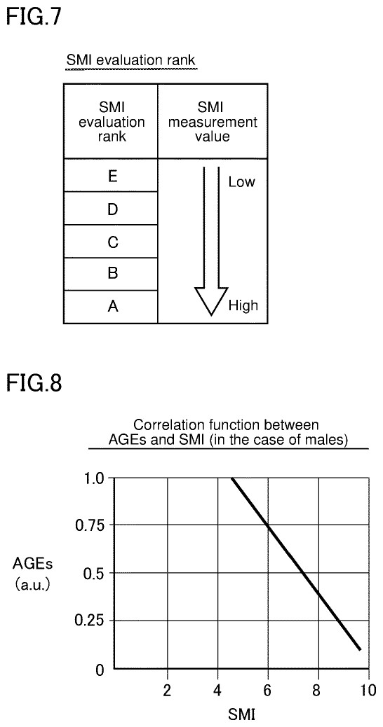

Referring to FIG. 7, the evaluation of SMI will be described. FIG. 7 is a diagram for explaining SMI evaluation ranks. As shown in FIG. 7, the prediction device 50 establishes a plurality of reference values for SMI measurement values in a stepwise manner and ranks SMI based on a comparison between SMI measurement values and the plurality of reference values. For example, five ranks from A to E (hereinafter also referred to as “SMI evaluation ranks”) have been established as the ranks to be assigned to SMI measurement values. The SMI evaluation rank of “A” gives the best SMI measurement value evaluation, and the SMI evaluation rank of “E” gives the worst SMI measurement value evaluation.

Note that any value may be set as the reference value for the SMI evaluation rank. Further, the reference value for the SMI evaluation rank may differ depending on the age of the subject, and may also differ depending on the gender of the subject.

### [Calculation of SMI Prediction Value Using AGEs Measurement Values]

Referring to FIG. 8 to FIG. 10, the calculation of the SMI prediction value using AGEs measurement values will be described.

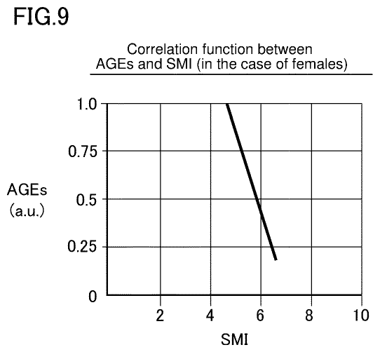

FIG. 8 and FIG. 9 are diagrams for explaining the correlation between AGEs and SMI. FIG. 8 shows a graph of the correlation when the subject is male, with SMI measurement values on the horizontal axis and AGEs measurement values on the vertical axis. FIG. 9 shows a graph of the correlation when the subject is female, with SMI measurement values on the horizontal axis and AGEs measurement values on the vertical axis.

As shown in FIG. 8 and FIG. 9, regardless of whether the subject is male or female, the smaller the AGEs measurement value, the larger the SMI measurement value, and the larger the AGEs measurement value, the smaller the SMI measurement value. Thus, there is a correlation between AGEs and SMI, and the prediction device 50 predicts an SMI measurement value at some future timing based on the time-series changes in the AGEs measurement value and the SMI measurement value, using the correlation.

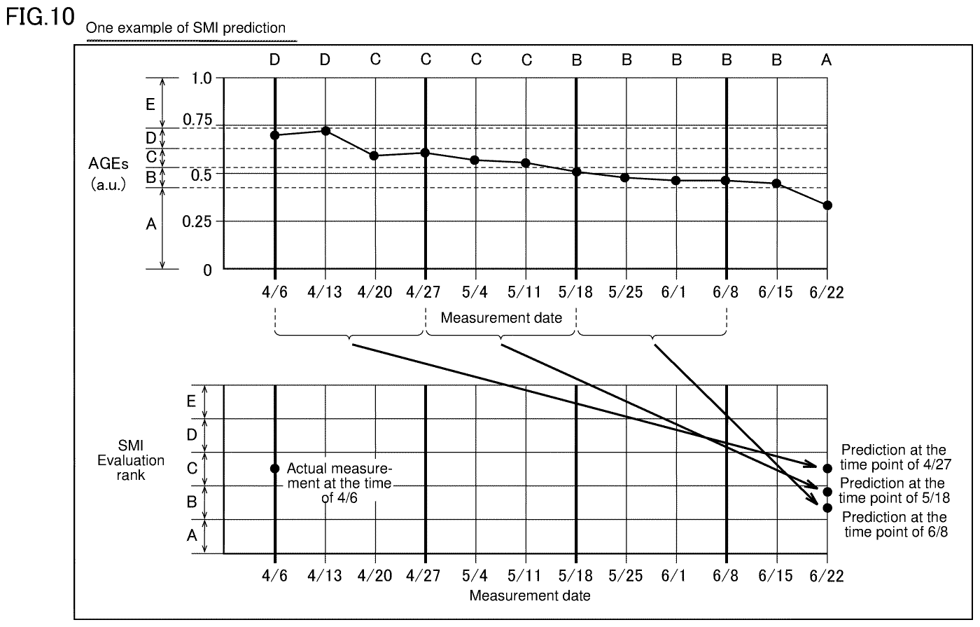

FIG. 10 is a diagram of one example of an SMI prediction. In FIG. 10, an example is shown in which the prediction device 50 calculates an SMI prediction value using the AGEs measurement values of the subject obtained every week over the period of 4/6-6/22 shown in FIG. 6. The upper part of FIG. 10 shows a graph showing the time-series changes in the AGEs measurement value of the subject, with the date on the horizontal axis and the AGEs measurement values on the vertical axis. The lower part of FIG. 10 shows a graph of the time-series change in the SMI evaluation rank of the subject, with the date on the horizontal axis and the SMI evaluation rank on the vertical axis.

As noted above, AGEs can change in roughly one to several weeks in response to improved lifestyle habits. For this reason, the subject measures AGEs periodically, usually every few weeks, using the AGEs measurement device 10. For example, the subject measures the AGEs every week over a period of 4/6-6/22 using the AGEs measurement device 10. The prediction device 50 obtains AGEs measurement values from the AGEs measurement device 10 every week over the period 4/6 to 6/22.

On the other hand, for SMI, SMI measurement values do not change as quickly as AGEs, typically requiring several months of improved lifestyle habits before changes begin to reflect in the measurement values. For this reason, the subject usually measures SMI every few months using the SMI measurement device 10. For example, if the subject measures SMI on 4/6 using the SMI measurement device 20, the next measurement of SMI is scheduled for 6/22, a few months later. The prediction device 50 obtains the SMI measurement value at the time point of 4/6 from the AGEs measurement device 20.

After obtaining the SMI measurement value at the time point of 4/6, the prediction device 50 calculates the SMI for the subject on 6/22, the next SMI measurement date, as the SMI prediction value at the subsequent prediction time point. Further, the prediction device 50 calculates the SMI prediction value based on the time-series changes in the AGEs measurement values obtained over a predefined period of time retroactively from the prediction time point.

For example, in the case of calculating the SMI prediction value for 6/22 at the time point of 4/27, the prediction device 50 reads the AGEs measurement values for 4/6, 4/13, 4/20, and 4/27, which were obtained over the past one week retroactively from 4/27, from the storage device 520. The prediction device 50 calculates the SMI prediction value at 6/22 based on the time-series changes in the AGEs measurement values collected weekly between 4/6 and 4/27, as well as the SMI measurement value obtained previously on 4/6. The prediction device 50 then calculates the SMI evaluation rank by comparing the calculated SMI prediction value with the standard value.

In the case of calculating the SMI prediction value for 6/22 at the time point of 5/18, the prediction device 50 reads the AGEs measurement values for 4/27, 5/4, 5/11, and 5/18, which were obtained over the past one week retroactively from 5/18, from the storage device 520. The prediction device 50 calculates the SMI prediction value at 6/22 based on the time-series changes in the AGEs measurement values collected weekly from 4/27 to 5/18, as well as the SMI measurement value obtained previously on 4/6. The prediction device 50 then calculates the SMI evaluation rank by comparing the calculated SMI prediction value with the standard value.

In the case of calculating the SMI prediction value for 6/22 at the time point of 6/8, the prediction device 50 reads the AGEs measurement values for 5/18, 5/25, 6/1, and 6/8, which were obtained over the past one week retroactively from 6/8, from the storage device 520. The prediction device 50 calculates the SMI prediction value at 6/22 based on the time-series changes in the AGEs measurement values collected weekly from 5/18 to 6/8, as well as the SMI measurement value obtained previously on 4/6. The prediction device 50 then calculates the SMI evaluation rank by comparing the calculated SMI prediction value with a standard value.

In the example shown in FIG. 10, the AGEs measurement values obtained from 4/6 to 6/8 are gradually improving (getting smaller), so the prediction device 50 predicts that the SMI evaluation rank at 6/22 at each prediction time point will also gradually improve. The SMI prediction values and the SMI evaluation ranks obtained in this way are stored in the viewing information table shown in FIG. 4 as SMI prediction information.

### [Display Example of Viewing Information]

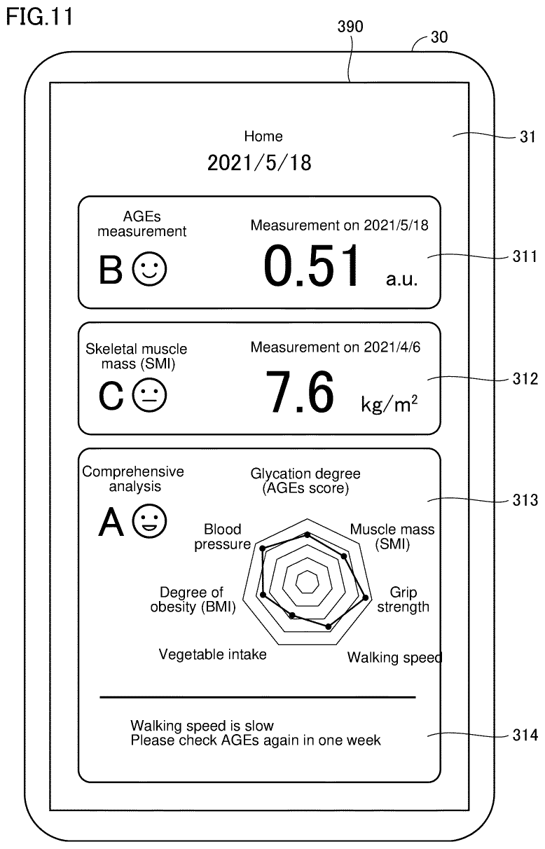

Referring to FIG. 11 to FIG. 15, a display example of viewing information will be described. FIG. 11 to FIG. 15 show examples of display screens on the user terminal 30 according to the embodiment.

When the user executes an application program for using the information provision service using the user terminal 30, the user terminal 30 displays a login screen on the display 390, which is not illustrated. When the user enters the user ID and the password on the login screen, the user terminal 30 outputs the user ID and the password to the prediction device 50. After the prediction device 50 authenticates the user based on the user ID and the password, the user terminal 30 displays the home screen 31 as shown in FIG. 11 on the display 390.

The home screen 31 includes an image 311 for viewing AGEs measurement information, an image 312 for viewing SMI measurement value and SMI prediction information, and an image 313 for viewing analysis information.

The image 311 shows the most recently measured AGEs measurement value and the AGEs evaluation rank corresponding to the AGEs measurement value. In this example, in the image 311, “0.51” is shown as the AGEs measurement value measured on May 18, 2021, and “B” is shown as the AGEs evaluation rank.

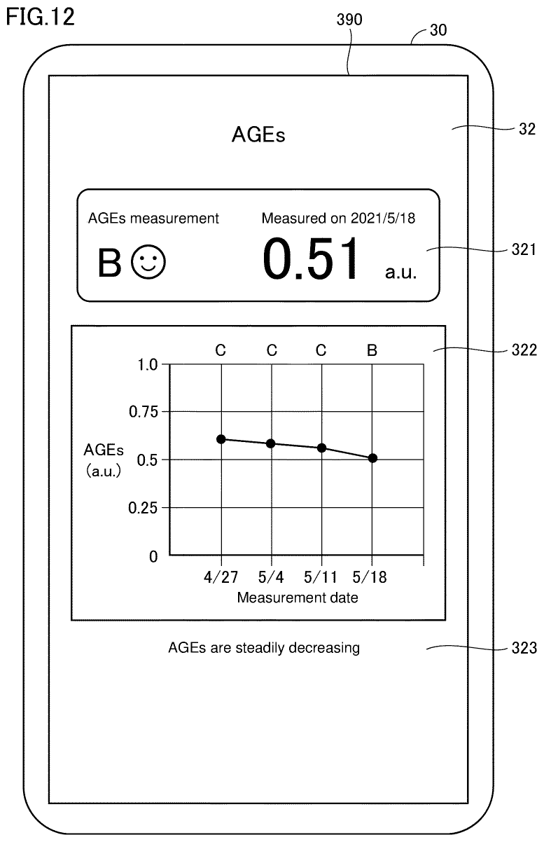

When the user selects (e.g., touches) the image 311, the user terminal 30 displays the AGEs viewing screen 32, as shown in FIG. 12, on the display 390.

The AGEs viewing screen 32 includes an image 321 showing the most recently measured AGEs measurement value, an image 322 showing the change in the AGEs measurement value over time in the past (for example, the past one week), and an image 323 showing comments about the AGEs measurement value. Thus, the user can view AGEs measurement information for the subject by referring to the AGEs viewing screen 32.

Returning to FIG. 11, the image 312 shows the most recently measured SMI measurement value and the SMI evaluation rank corresponding to the SMI measurement value. In this example, in the image 312, “7.6” is shown as the SMI measurement value measured on Apr. 6, 2021, and “B” is shown as the SMI evaluation rank.

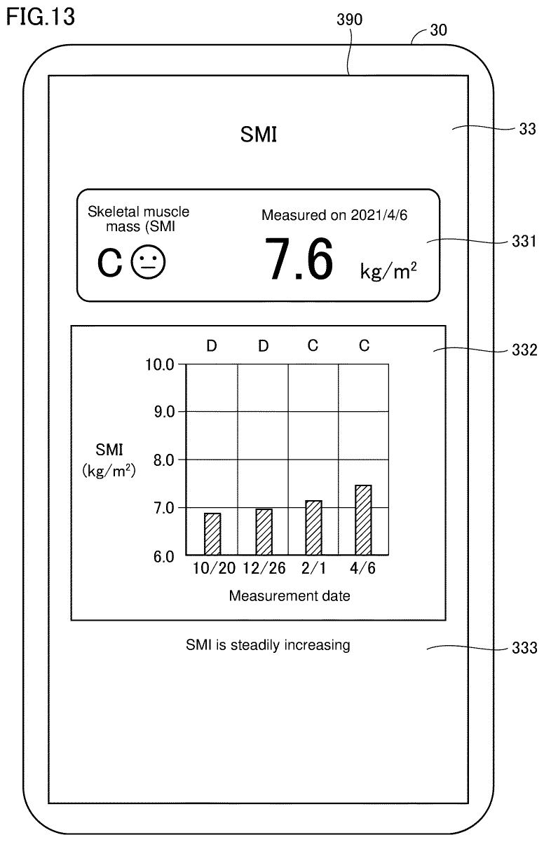

When the user selects the image 312, the user terminal 30 displays the SMI viewing screen 33 as shown in FIG. 13 on the display 390.

The SMI viewing screen 33 includes an image 331 showing the most recently measured SMI measurement value, an image 332 showing the time-series change in the SMI measurement values in the past (e.g., the past six months), and an image 333 showing comments about the SMI measurement value. As described above, the user can view the SMI measurement information of the subject by referring to the SMI viewing screen 33.

Returning to FIG. 11, the image 313 includes the analysis results obtained by analyzing the health status of the subject based on AGEs measurement values and an SMI measurement value, etc., as well as comments about the analysis result.

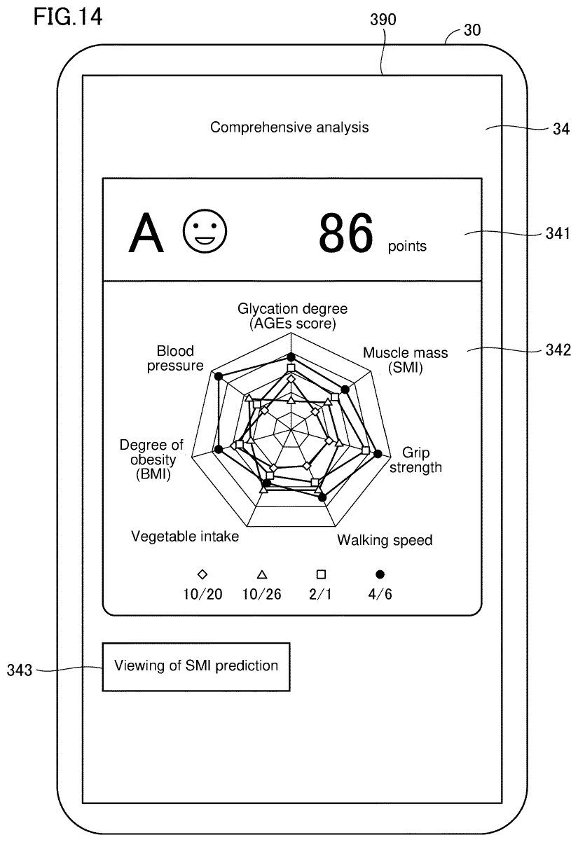

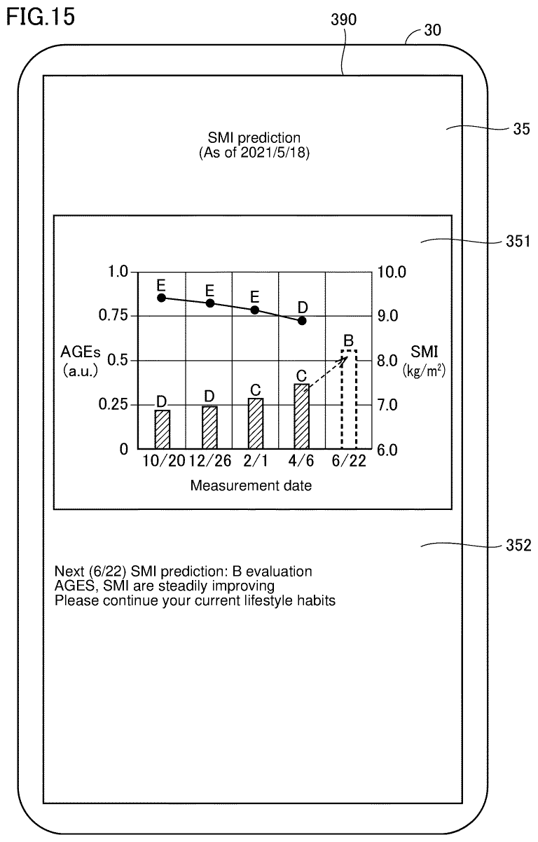

When the user selects the image 313, the user terminal 30 displays the comprehensive analysis screen 34, as shown in FIG. 14, on the display 390.

The comprehensive analysis screen 34 includes an image 341 showing the score and the evaluation rank corresponding to the analysis result and an image 342 showing the analysis results in a radar chart. Thus, by referring to the comprehensive analysis screen 34, the user can view the analysis information on the subject.

Further, the comprehensive analysis screen 34 includes an image 343 for viewing an SMI prediction value. When the user selects the image 343, the user terminal 30 displays the SMI prediction screen 35 as shown in FIG. 15 on the display 390.

The SMI prediction screen 35 includes an image 351 showing the SMI prediction value at the next SMI measurement date (6/22 in this example) calculated at the current time (as of 5/18 in this example) and an image 352 showing advice regarding the subject's lifestyle habits based on the SMI prediction value. In this example, in the image 351, as a result of predicting SMI at 5/18, the SMI evaluation rank is shown to improve from a “C” to a “B” at 6/22. Further, in the image 352, as the advice regarding lifestyle habits, a comment is indicated that the subject should continue their current lifestyle habits because their AGEs and SMI are steadily improving. Thus, by referring to the SMI prediction screen 35, the user can view SMI prediction information such as SMI prediction values, the SMI evaluation rank corresponding to the SMI prediction value, and lifestyle habits advice based on the SMI prediction.

### [Processing of Prediction Device]

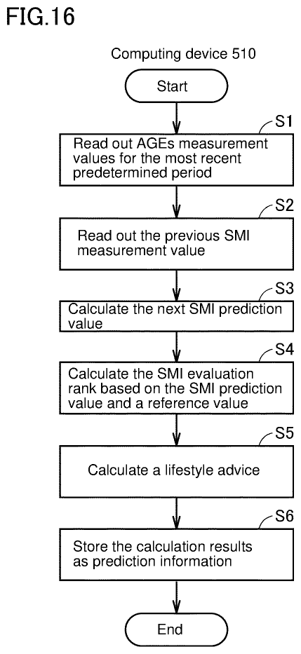

Referring to FIG. 16, the processing of the prediction device 50 will be described. FIG. 16 is a flowchart of prediction processing executed by the prediction device 50 of an embodiment. The processing step (hereafter referred to as “S”) shown in FIG. 16 is realized by the computing device 510 executing the prediction program 521.

As shown in FIG. 16, the prediction device 50 reads AGEs measurement values from the storage device 520 for the most recent predetermined period of time retroactively from the present, which is the prediction time point (S1). The prediction device 50 reads the previously obtained SMI measurement values from the storage device 520 (S2).

The prediction device 50 calculates the SMI prediction value for the next measurement date based on the time-series changes in the read AGEs measurement values and the read SMI measurement value (S3). The prediction device 50 calculates the SMI evaluation rank corresponding to the SMI prediction value by comparing the calculated SMI prediction value with a reference value (S4). Further, the prediction device 50 calculates the lifestyle advice based on the SMI evaluation rank (S5).

The prediction device 50 stores the SMI prediction value calculated in S4 and the lifestyle habits advice calculated in S5 in the storage device 520 by storing them as prediction information in the viewing information table in FIG. 4 (S6).

As described above, according to the prediction device 50 of this embodiment, the SMI prediction value and the SMI evaluation rank in the future are calculated based on the time-series changes in the AGEs measurement values obtained in the past and the SMI measurement value obtained in the past. By viewing the SMI prediction value and the SMI evaluation rank using the user terminal 30, the subject (user) can easily maintain motivation to continue improving lifestyle habits even while observing not only the AGEs measurement values that are repeatedly obtained over a relatively short period of time, but also the SMI measurement values that are repeatedly obtained over a relatively long period of time.

### [Modifications of Prediction Device of Embodiment]

Although the prediction device 50 of the embodiment has been described above, various further modifications and applications are possible in the prediction device 50 of the embodiment. Hereinafter, only those components of the prediction device 50 that differ from those of the embodiment's prediction device 50 will be described.

The prediction device 50 according to the embodiment calculated the SMI prediction value on the next SMI measurement date. However, the prediction device 50 may calculate an SMI prediction value at any timing as long as it is the timing after the present, which is the prediction time point.

The prediction device 50 according to the embodiment calculated an SMI prediction value using AGEs measurement values obtained in the past one week retroactively from the present, which is the prediction time point. However, the prediction device 50 need only calculate the SMI prediction value using the AGEs measurement values that have already been obtained at the present, which is the prediction time point. For example, it may calculate the SMI prediction value using all previously obtained AGEs measurement values.

The prediction device 50 according to the embodiment obtained AGEs measurement values directly from the AGEs measurement value 10. However, the subject may input AGEs measurement values obtained by the AGEs measurement value 10 from their terminal (e.g., the user terminal 30), and the prediction device 50 may obtain AGEs measurement values from the subject's terminal (e.g., the user terminal 30).

The prediction device 50 according to the embodiment obtained the SMI measurement value directly from the SMI measurement value 20. However, the subject may input an SMI measurement value obtained by the SMI measurement value 20 from their terminal (e.g., the user terminal 30), and the prediction device 50 may obtain SMI measurement value from the subject's terminal (e.g., the user terminal 30).

The prediction device 50 according to the embodiment provided viewing information, such as an SMI prediction value, to the user by displaying it on the display 390 of the user terminal 30, as shown in FIG. 11 to FIG. 15. However, the prediction device 50 may output a paper showing viewing information, such as an SMI prediction value, to an external source using a printer.

### ASPECTS

It would be understood by those skilled in the art that the plurality of exemplary embodiments described above is specific examples of the following aspects.

### (Item 1)

A prediction device according to one aspect is provided with a computing device and a communication device connected to the computing device in a communicable manner. The communication device is configured to acquire a first measurement value of advanced glycation end-products of the subject a plurality of times in a time-series manner, and acquire a second measurement value of the skeletal muscle mass of the subject. The computing device calculates prediction information regarding a prediction value of the second measurement value, based on the first measurement value acquired a plurality of times in a time-series manner and the second measurement value.

According to the prediction device as recited in the above-described Item 1, the subject can know the prediction information about the SMI prediction value, based on the time-series change in the AGEs measurement values in addition to the SMI measurement value. With this, it is expected that it becomes easier to maintain motivation to continue to improve lifestyle habits while observing the SMI.

### (Item 2)

The communication device is configured to acquire the first measurement values periodically at a first interval, and acquire the second measurement values periodically at a second interval longer than the first interval.

According to the prediction device as recited in the above-described Item 2, it is easier to maintain the motivation to continuously improve lifestyle habits even while observing not only AGEs measurement values but also SMI measurement values that are repeatedly obtained over a longer period of time than AGEs measurement values.

### (Item 3)

The computing device calculates the prediction information, based on time-series changes in the first measurement values acquired in a predetermined period within an acquisition period of the first measurement values.

According to the prediction device as recited in the above-described Item 3, it is possible for a subject to obtain prediction information regarding the SMI prediction value by measuring AGEs over a predetermined period of time.

### (Item 4)

The computing device calculates the prediction information, based on time-series changes in the first measurement values acquired in a predetermined period that is most recent retroactively from a prediction time point, within the acquisition period.

According to the prediction device as recited in the above-described Item 4, it is possible for a subject to obtain prediction information regarding the SMI prediction value by measuring AGEs over the most recent predetermined period retroactively from the prediction time point.

### (Item 5)

The computing device calculates, as the prediction information, an evaluation result of the prediction value based on a comparison with a reference value.

According to the prediction device as recited in the above-described Item 5, by observing the evaluation result of the SMI prediction value (the SMI evaluation rank), the subject can more easily maintain motivation to continue improving lifestyle habits through SMI.

### (Item 6)

The computing device calculates, as the prediction information, an advice regarding lifestyle habits of the subject based on the prediction value.

According to the prediction device as recited in the above-described Item 6, the subject can easily maintain motivation to continue improving lifestyle habits while viewing the advice on the subject's lifestyle habits based on the SMI prediction value.

### (Item 7)

A prediction method according to one aspect, comprising:

a step of acquiring a first measurement value of the advanced glycation end-products of the subject a plurality of times in a time-series manner;
a step of acquiring a second measurement value of the skeletal muscle mass of the subject; and
a step of calculating prediction information regarding a prediction value of the second measurement value, based on the first measurement value acquired a plurality of times in a time-series manner and the second measurement value.

According to the prediction method as recited in the above-described Item 7, the subject can know prediction information regarding the SMI prediction value based on the time-series changes in the AGEs measurement values in addition to the SMI measurement value. With this, it is expected that it becomes easier to maintain motivation to continue improving lifestyle habits while observing the SMI.

### (Item 8)

A prediction program according to one aspect being configured to make a computing device execute:

a step of acquiring a first measurement value of advanced glycation end-products of the subject a plurality of times in a time-series manner; and
a step of acquiring a second measurement value of the skeletal muscle mass of the subject; and
a step of calculating prediction information regarding a prediction value of the second measurement value, based on the first measurement value acquired a plurality of times in a time-series manner and the second measurement value.

According to the prediction program as recited in the above-described Item 8, the subject can know the prediction information about the SMI prediction value based on the time-series changes in the AGEs measurement values in addition to the SMI measurement value. With this, it is expected that it becomes easier to maintain motivation to continue improving lifestyle habits while observing the SMI.

### DESCRIPTION OF REFERENCE SYMBOLS

1: Prediction System
10: AGEs measurement device
20: SMI measurement device
11: Measurement unit
12, 390: Display
13: Communication unit
30: User terminal
31: Home screen
32: AGEs viewing screen
33: SMI viewing screen
34: Comprehensive analysis screen
35: Prediction screen
50: Prediction device
311, 312, 313, 321, 322, 323, 331, 332, 333, 341, 342, 343, 351, 352: Image
510: Computing device
520: Storage device
521: Prediction program
522: User identification information
523: Viewing information
530: Communication device

1. A prediction device for predicting a skeletal muscle mass of a subject, comprising:
a computing device; and
a communication device connected to the computing device in a communicable manner,
wherein the communication device is configured to
acquire a first measurement value of advanced glycation end-products of the subject a plurality of times periodically at a first interval, and
acquire a second measurement value of the skeletal muscle mass of the subject periodically at a second interval longer than the first interval, and
wherein the computing device calculates prediction information regarding a prediction value of the second measurement value, based on the first measurement values acquired a plurality of times and the second measurement value.

2. (canceled)

3. The prediction device as recited in claim 1,
wherein the computing device calculates the prediction information, based on time-series changes in the first measurement values acquired in a predetermined period within an acquisition period of the first measurement values.

4. The prediction device as recited in claim 3,
wherein the computing device calculates the prediction information, based on the time-series changes in the first measurement values acquired in the predetermined period that is most recent retroactively from a prediction time point, within the acquisition period.

5. The prediction device as recited in claim 1,
wherein the computing device calculates, as the prediction information, an evaluation result of the prediction value based on a comparison with a reference value.

6. The prediction device as recited in claim 1,
wherein the computing device calculates, as the prediction information, an advice regarding lifestyle habits of the subject based on the prediction value.

7. A prediction method for predicting a skeletal muscle mass of a subject by a computing device, comprising:
a step of acquiring a first measurement value of advanced glycation end-products of the subject a plurality of times periodically at a first interval;
a step of acquiring a second measurement value of the skeletal muscle mass of the subject periodically at a second interval longer than the first interval; and
a step of calculating prediction information regarding a prediction value of the second measurement value, based on the first measurement values acquired a plurality of times and the second measurement value.

8. (canceled)
